(19) United States
(12) Patent Application Publication
(10) Pub. No.: US 2024/0016652 A1
(43) Pub. Date: Jan. 18, 2024

(54) INTEGRATED OSTOMY GARMENT

(71) Applicant: Jeff Calkins, Seattle, WA (US)

(72) Inventor: Jeff Calkins, Seattle, WA (US)

(21) Appl. No.: 18/329,500

(22) Filed: Jun. 5, 2023

Related U.S. Application Data
(60) Provisional application No. 63/348,980, filed on Jun. 3, 2022.

(51) Int. Cl. A61F 5/449; A61F 5/03

(52) U.S. Cl. CPC A61F 5/449; A61F 5/03

(57) ABSTRACT

An integrated ostomy garment is disclosed. The garment includes a first upper layer having a shoulder portion and an internal pocket on the front side of the first upper layer. The internal pocket is capable of supporting an ostomy bag when the garment is worn and is accessible externally via side openings in the side seams of the first upper layer. The garment further includes a second upper layer connected to the first upper layer, also having a shoulder portion, as well as a lower portion. The garment further includes a waist portion connected to the second upper layer and the lower layer, which includes one or more apertures for accessing the wearer's stoma and through which the wearer's ostomy bag is placed when the garment is worn. The waist portion may further include one or more retention elements for supporting the ostomy bag, and may function as hernia belt.

### CROSS-REFERENCE TO RELATED APPLICATION

This disclosure claims benefit of U.S. Provisional Application No. 63/348,980, titled “SECOND CHANCE′ SUIT,” filed on Jun. 3, 2022, the disclosure which is incorporated herein by reference in its entirety.

### FIELD OF THE INVENTION

This disclosure relates to an integrated medical garment for persons with stomas, such as persons that have undergone a colostomy procedure.

### BACKGROUND

Colostomies and ileostomies are both bowel division surgeries that permit stool to leave the body by bypassing a damaged portion of the intestine. They are used to either provide time for the damaged bowel to heal, as in the case of a temporary colostomy or ileostomy; or to remove part of the bowel due to disease or damage, as in the case of a permanent colostomy or ileostomy. Ileostomy involves the distal portion of the small intestine, while colostomy involves the large intestine. Urostomy, another ostomy procedure, involves bypass of the bladder for patients in need of bladder removal. In each ostomy instance, an opening, called a stoma, is created in the patient's abdomen. Typical reasons for ostomy procedures include cancer, inflammatory disease, blockages, and infection. Persons who have had an ostomy surgery are sometimes referred to as “ostomates.”

Colostomies are instructive. A colostomy is a surgical procedure in which a portion of the colon or rectum is either temporarily bypassed, as in a temporary colostomy, or permanently removed, as in a permanent or “end” colostomy. For permanent colostomies, the end of the colon is brought through the abdominal wall, where it is turned under like a cuff. The colon edges are stitched to the skin of the abdominal wall, forming the stoma. Stool passes from the stoma into an ostomy bag or pouch attached to the abdomen. Ostomates with a permanent colostomy will need to wear an ostomy bag for the rest of their lives.

Because the ostomy bag collects stool (or, in the case of a urostomy, urine), the bag naturally becomes heavier as it fills. In some instances, the ostomy bag is taped directly to the ostomate's abdomen. As the bag fills and grows heavier, this causes the tape to pull on the ostomate's skin, causing pain and discomfort. The tape itself can cause pain and discomfort by reacting adversely with the ostomate's skin. In other instances, the ostomate may choose to wear an ostomy belt around their waist, with which the ostomy bag can be held rather than being taped to the ostomate's skin. But again, as the bag fills and becomes heavier, the weight of the bag pulls the belt downward. As the belt slips, the belt itself can abrade the ostomate's skin. As gravity pulls the bag downward in either case, it can pull on the ostomate's stoma, causing tearing or detachment of the ostomy bag from the stoma, potentially damaging the stoma. This is painful and uncomfortable for the ostomate, and reduces the ostomate's overall quality of life.

Another unpleasant problem that can arise from wearing an ostomy bag is pancaking. Ostomy pancaking occurs when stool overflows from the stoma, leaving a thick, pancake-like mass of stool at the top of the bag. In some instances, the pancaking causes the bag to be forced off the body entirely, referred to as “pancake blowouts.” Pancaking is uncomfortable and embarrassing for the affected ostomate, and can happen unexpectedly, decreasing the ostomate's overall quality of life.

Ostomates often face other issues related to having had an ostomy. For instance, a colostomy will often lead to weakened abdominal muscles, increasing the risk of hernias, which are bulging of tissue or an organ through an opening in the abdomen. Doctors often recommend that ostomates wear a hernia belt to help support their abdominal muscles and prevent hernias developing. Wearing such a belt can be difficult though, as the belt typically needs to be worn around the portion of the abdomen in which the stoma is formed, such that the ostomy bag must be pressed between the belt and the ostomate's abdomen. Activity can cause sweat to form between the bag and the ostomate's abdomen, which can be uncomfortable and accompanied by an unpleasant odor. Hernia belts specifically for ostomates are available, such as the Stealth Belt® which includes a hole through which the ostomy bag is pulled and a zip-up pouch in which the bag sits while the belt is worn. However, these products still suffer from the same problem as a traditional ostomy belt, for as the ostomy bag fills and becomes heavier, the hernia belt is pulled downward by gravity, pulling on the stoma and causing pain and discomfort for the ostomate. The ostomy bag may also not be readily accessible in such belts, such that the belt must be entirely removed to access and change the bag, which can be difficult and time consuming, as well as a constant reminder to the ostomate of the differences they now experience as a result of their procedure.

Ostomy garments are a potential option for ostomates. Such garments typically consist of underwear-like garments that include a pocket for holding the ostomy bag. But these garments suffer from the same problems as hernia belts and traditional ostomy belts. That is, as the ostomy bag fills and becomes heavier, gravity pulls the garment downward, abrading the skin and pulling on the stoma and causing pain and discomfort for the ostomate. Once again, the ostomy bag may not be readily accessible in these garments, such that the garment must be removed entirely to access and change the bag. These garments are not designed to have the appearance of normal clothing, and as a result can lead the wearer to be self-conscious or otherwise interfere with the wearer's comfort and confidence. And where such garments are form-fitting, as undergarments typically are, the garment may protrude from the ostomate's body as the bag fills, causing unsightly bulges under the ostomate's clothing.

Approximately 725,000 to 1,000,000 Americans have an ostomy. Those numbers are expected to increase as diseases like colorectal cancer and inflammatory bowel disease increase in incidence. Indeed, the global market for ostomy bags is forecast to reach nearly $4.1 billion by 2027. The factors driving market growth include increasing incidence of chronic conditions such as colorectal cancer, bladder cancer, Chrohn's disease, ulcerative colitis, and inflammatory bowel disease. The incidence of these sorts of diseases is increasing in younger patients—people with many active years ahead of them, but for the impediments created by their ostomy.

Ostomy surgery provides a new lease on life for people of all ages, allowing ostomates to go on to live active and healthy lives for many additional years. However, existing options for ostomates fall short. Like any other person, ostomates want to feel comfortable and secure while being active, or just feel “normal” in their own skin when going about their day or heading out on the town. Yet available ostomy belts and garments fall short of this need for “normal,” proving impractical, uncomfortable, and serving as a constant reminder of the differences the ostomate now faces in their next stage of life.

Embodiments of the invention address these and other shortcomings of the prior art.

### BRIEF DESCRIPTION OF THE DRAWINGS

FIG. 1 is a front view of a complete integrated ostomy garment according to embodiments of the invention, as worn for purposes of context only.

FIG. 2 is a rear view of a complete integrated ostomy garment according to embodiments of the invention, as worn for purposes of context only.

FIG. 3 is a front angled view of a complete integrated ostomy garment according to embodiments of the invention, as worn for purposes of context only.

FIG. 4 is a front view of a portion of an integrated ostomy garment according to embodiments of the invention, as worn for purposes of context only.

FIG. 5 is a rear view of a portion of an integrated ostomy garment according to embodiments of the invention, as worn for purposes of context only.

FIG. 6 is a partially exploded view of a complete integrated ostomy garment according to embodiments of the invention, as worn for purposes of context only.

FIG. 7 is a close-up front view of a portion of an integrated ostomy garment according to embodiments of the invention, as worn for purposes of context only.

### DETAILED DESCRIPTION OF THE EMBODIMENTS

The embodiments described herein provide an integrated garment for persons with stomas, such as persons that have undergone a colostomy procedure. The disclosed ostomy garment provides stylish concealment of ostomy appliances while looking like a standard undergarment in a wardrobe. These embodiments are wearable all day, whether the wearer is active or casual, sleeping, or engaged in intense physical activity. By being a completely integrated garment with an upper shirt connected to a bottom, the ostomate need only have one undergarment without additional belts, bands, or shields. The garment's internal pocket conceals and comfortably supports the weight of appliances as they fill. The garment also includes a compression waistband that applies support for hernias or protection of potential hernias due to scarring and weak abdominal muscles, while also adding pressure to the adhesive used to attach the ostomy bag for extra security. The upper portion of the garment provides extra support over the wearer's shoulders such that the garment's waistband does not ride down or tug at appliances, while pockets along the side seam of the upper portion allow for discrete access to the ostomy bag. These and other features of the disclosed garment provide the wearer with the feeling of confidence, security, and normalcy as they navigate through their new chapter, and can be incorporated into any lifestyle, from extreme and active to comfortable and lazy. In this manner, the embodiments described herein have the potential to give diagnosed, recovering, and recovered patients hope and drive to embrace their new chapter, by providing a comfortable, secure, and discrete garment.

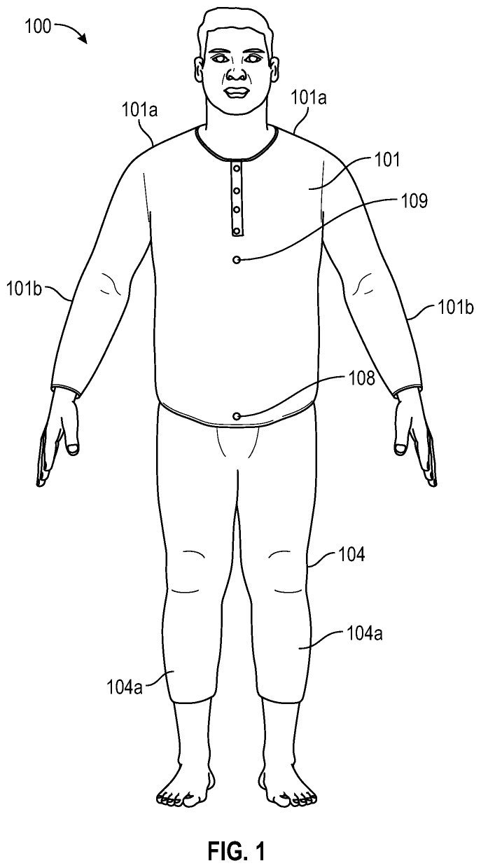

FIGS. 1-7 show different views of an integrated ostomy garment 100 according to an embodiment of the invention.

As shown in FIGS. 1-5, in an embodiment the integrated garment 100 includes a first upper layer 101, a second upper layer 102, a waist portion 103, and a lower portion 104.

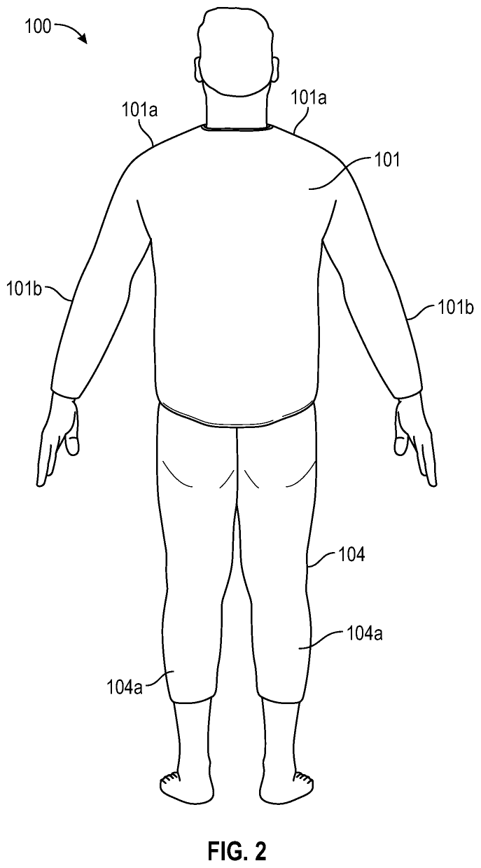

As shown in FIGS. 1-2, the first upper layer 101 includes at least one shoulder portion 101a, such that the first upper layer 101 is supported by the wearer's shoulder when the integrated garment 100 is being worn. In an embodiment, the first upper layer 101 includes two shoulder portions 101a. In an embodiment, the first upper layer 101 also includes at least one sleeve portion 101b. In an embodiment, the first upper layer 101 includes two sleeve portions 101b. The sleeve portions 101b may be long sleeves, short sleeves, mid-length sleeves, or any other known sleeve style or configuration. In another embodiment, the first upper layer 101 does not include any sleeve portions 101b, such that the first upper layer 101 is in the style of a sleeveless shirt, such as a muscle shirt or a tank top.

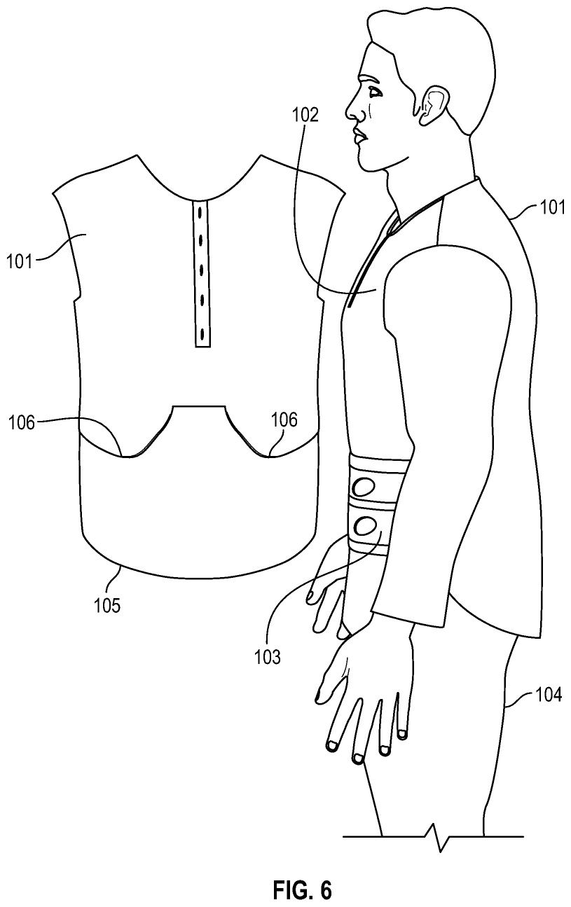

As better seen in FIG. 6, in an embodiment the first upper layer 101 further includes one or more internal pockets 105 formed on the internal front side of first upper layer 101 (the face of upper layer 101 closest to the wearer's body when the garment 100 is being worn). Internal pocket 105 is capable of receiving an ostomy bag through one or more top openings 106 located substantially at the top of the internal pocket 105. The internal pocket 105 and the first upper layer 101 are sewn or otherwise connected substantially at the bottom hem of the first upper layer 101, such that when an ostomy bag is received in the internal pocket 105, the ostomy bag is supported by resting substantially at the bottom of the internal pocket 105.

In an embodiment, internal pocket 105 includes two top openings 106, one located substantially on the lefthand side of the garment 100 and one located substantially on the righthand side of the garment 100. In this manner, the wearer can readily slip their ostomy bag into the internal pocket, regardless of which side of their abdomen their stoma is located. In an embodiment, the one or more top openings 106 of internal pocket 105 do not include any closure element. In another embodiment, the one or more top openings 106 of internal pocket 105 include one or more closure elements, such as a button-and-buttonhole closure, a hook-and-eye closure, a snap closure, a VELCRO® closure, a zipper closure, or any other known closure type.

In an embodiment, internal pocket 105 may be formed of a single piece of material forming one large pocket spanning the width of the front side of first upper layer 101, which can be but need not be sewn substantially in the middle to form two pockets inside the internal pocket 106. In another embodiment, internal pocket 105 may be formed of two or more pieces of material sewn or otherwise connected to the inside face of first upper layer 101.

In an embodiment, additional elements may be included substantially inside internal pocket 105 to increase the garment's versatility and wearability. For example, a retention element, such as a strap (not shown), may be included substantially inside internal pocket 105, which may be used to hold the wearer's ostomy bag in place when the bag is substantially inside internal pocket 105. A removable liner (not shown) may also be included substantially inside internal pocket 105. The liner may be made of different materials depending on its purpose. For example, the liner may be made of a charcoal fabric to aid in reducing odor from the wearer's ostomy bag. Alternatively or in addition, the liner may be made of an antimicrobial and/or antibacterial fabric to aid in microbial and/or bacterial growth inside the internal pocket 105, which can result from an ostomy bag. Alternatively or in addition, the liner may be made of a waterproof or water resistant fabric to aid in reducing or eliminating unsightly spills from the ostomy bag, such as occur in the case of a pancake blowout. One or more removable liners may be used. In another embodiment, the one or more liners may not be removable, but instead may be permanently affixed substantially inside internal pocket 105.

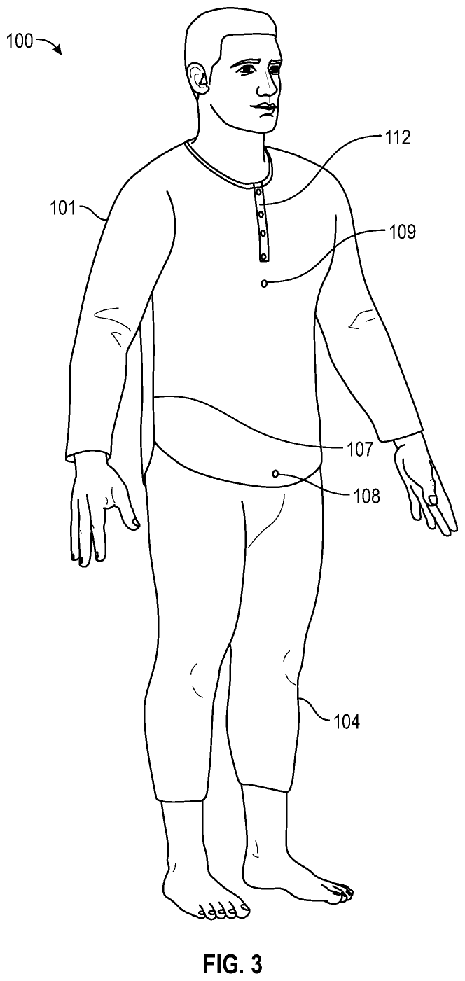

As shown in FIG. 3, in an embodiment the first upper layer 101 further includes at least one side opening 107 located substantially toward the bottom hem and along the side seam of the first upper layer. The garment may include only one side opening 107 on only one side of the first upper layer 101, or may include two side openings 107, one on both sides of the first upper layer 101. The side openings 107 may run substantially the height of the internal pocket 105 at the side seam of the first upper layer 101. By including the side openings 107 substantially along the side seams of the first upper layer 101, the visibility of the side openings 107 is minimized, providing the garment 100 with a more normal feeling and appearance. The side openings 107 enable the wearer to place their hand through the opening and into the internal pocket 105 to access their ostomy bag from the external side of the first upper layer 101, allowing the wearer to quickly and discretely check their bag without having to lift up their shirt to access the bag. In this manner, the wearer may access and check their ostomy bag without having to raise the hem of their shirt to access the bag from the top opening 106 of the internal pocket 105, minimizing the need for the wearer to expose themself and increasing the wearer's comfort, confidence, and overall quality of life.

In an embodiment, the one or more side openings 107 do not include any closure element. In another embodiment, the one or more side openings 107 include one or more closure elements, such as a button-and-buttonhole closure, a hook-and-eye closure, a snap closure, a VELCRO® closure, a zipper closure, or any other known closure type.

As shown in FIGS. 1 and 3, in an embodiment, the first upper layer 101 further includes a placket 112 located substantially in the center front of the first upper layer 101. Placket 112 may be formed at a neckline of the first upper layer 101 and substantially in the middle of the front side of the first upper layer 101. Placket 112 may include one or more buttons running substantially the length of placket 112. In this manner, the center front of the first upper layer 101 may be opened or closed, partially or fully, as desired by the wearer of garment 100, adding to the garment's versatility and comfort. Placket 112 may also aid in putting on the garment 100, as the placket 112 can be opened fully, enabling the wearer to step into the garment 100 and pull the garment 100 up such that the waist portion 103 is positioned over the wearer's midsection. In another embodiment, the first upper layer 101 may not include a placket 112. In another embodiment, the center front of the first upper layer 101 may include a different closure type, such as a zipper closure, a snap closure, a hook-and-eye closure, or a VELCRO® closure, in substantially the location of the placket 112.

In an embodiment, the first upper layer 101 further includes a fastening apparatus 113 consisting of a first closure element 108 and a second closure element 109. The first closure element 108 may be located substantially in the middle of a lower hemline of the first upper layer 101, while the second closure element 109 may be located substantially in the middle of the upper half of the external front side of the first upper layer 101. In an embodiment, first closure element 108 is a buttonhole, while second closure element 109 is a corresponding button. In an embodiment first upper layer 101 that includes a button placket 112, any of the buttons of the placket 112 can serve as the second closure element 109. Alternatively, first closure element 108, located substantially in the middle of the lower hemline, may be a button, while first closure element 109, located substantially in the middle of the upper half of the external front side of the first upper layer 101, may be a corresponding buttonhole. In another embodiment, the first and second closure elements 108, 109, making up the fastening apparatus 113, may be any other known form of garment closure, such as a snap closure, a hook-and-eye closure, a VELCRO® closure, or a zipper closure.

Fastening apparatus 113 allows the lower hemline of the first upper layer 101 to be raised and secured in place to the external front side of the first upper layer 101 via the first and second closure elements 108, 109 when necessary or desirable to do so, such as when using the bathroom or otherwise desiring access to the ostomy bag for removal or replacement. The lower hemline of the first upper layer 101 can thus be raised and held in place by the fastening apparatus 113, freeing the wearer's hands (or teeth) from having to hold up the lower hemline of the first upper layer 101 in order to access the ostomy bag for removal or replacement. In this manner, the first upper layer 101 can be longer than the second upper layer 102, extending past the waist portion 103, such that the overall garment 100 retains a more standard garment appearance rather than looking like a medical garment. By keeping the first upper layer 101 loose and casual in nature, the bag is also better concealed when the garment is in use. In another embodiment, multiple fastening apparatuses 113 may be included on the garment 100.

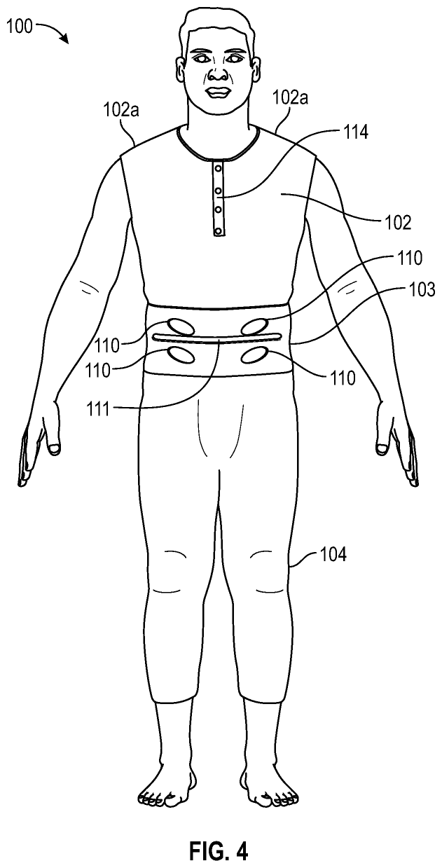

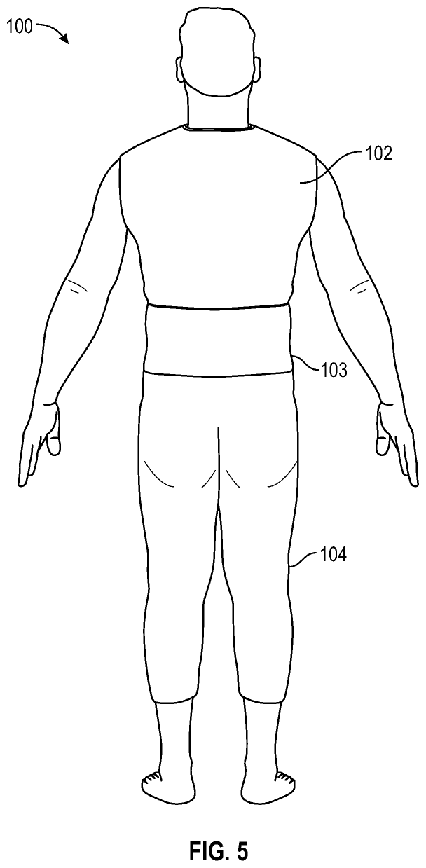

As shown in FIGS. 4-5, in an embodiment the second upper layer 102 of the integrated garment 100 includes at least one shoulder portion 102a, such that the second upper layer 102 is supported by the wearer's shoulder when the integrated garment 100 is being worn. In an embodiment, the second upper layer 102 includes two shoulder portions 102a. In an embodiment, such as the one shown in FIG. 4, the second upper layer 102 does not include any sleeve portions, such that the second upper layer 102 is in the style of a sleeveless shirt, such as a muscle shirt or a tank top. In another embodiment, the upper layer 102 may include one or two sleeve portions, which may be long sleeves, short sleeves, mid-length sleeves, or any other known sleeve style or configuration.

In an embodiment, the second upper layer 102 further includes a placket 114 located substantially in the center front of the second upper layer 102. Placket 114 is formed at a neckline of the second upper layer 102 and substantially in the middle of the front side of the second upper layer 102. Placket 114 may include one or more buttons running substantially the length of placket 114. In this manner, the center front of the second upper layer 102 may be opened or closed, partially or fully, as desired by the wearer of garment 100, adding to the garment's versatility and comfort. Placket 114 may also aid in putting on the garment 100, as the placket 114 can be opened fully, enabling the wearer to step into the garment 100 and pull the garment 100 up such that the waist portion 103 is positioned over the wearer's midsection. In another embodiment, the second upper layer 102 may not include a placket 114. In another embodiment, the center front of the second upper layer 102 may include a different closure type, such as a zipper closure, a snap closure, a hook-and-eye closure, or a VELCRO® closure, in substantially the location of the placket 114.

By utilizing two layers for the upper portion of the garment 100—the first upper layer 101 and the second upper layer 102—the garment 100 provides support for the ostomy appliances by distributing weight over the wearer's shoulders and thus reducing the weight on the wearer's stoma. The first upper layer 101 and the second upper layer 102 are substantially connected, for instance, by being sewn together at the shoulder seams and/or along the side seams of second upper layer 102, which are shorter than the side seams of the first upper layer 101. By using two layers for the upper portion of the garment 100, the waist portion 103 is connected to the second upper layer 102, which is hidden when the garment is in use by the first upper layer 101. In this manner, the waist portion 103 is fully supported through its connection to the second upper layer 102, while the first upper layer 101 fully covers the waist portion 103, providing a casual fit and a look as close to a regular shirt as possible. The overall garment thus provides ostomy bag concealment and support, and does not shift or ride down while in use, reducing or eliminating abrasion of the wearer's skin, as well as providing additional support for the wearer in the abdominal region. The garment accordingly provides wearers with a level of comfort, security, and confidence in their everyday lives while engaging in a myriad of activities—everything from lounging to driving to exercising.

As also shown in FIG. 4, the integrated garment 100 further includes a waist portion 103. The waist portion 103 is substantially connected to the second upper layer 102, for instance, by being sewn to the second upper layer 102. The waist portion 103 essentially forms a band around the midsection of the wearer when the garment is in use, and is worn around the portion of the wearer's abdomen where their stoma is located.

In an embodiment, the waist portion 103 is formed as a separate piece from the second upper layer 102, which is connected to the second upper layer 102, such as being sewn to the second upper layer 102. The waist portion 103 may be formed from a different type of material than the type of material used to form the second upper layer 102. For instance, the waist portion 103 may be formed from a type of material having compression characteristics, such as spandex, while the second upper layer 102 may be formed from a type of material having comfort characteristics, such as a jersey cotton fabric, or from a type of material having wicking characteristics, such as a polyester fabric. In this manner, the waist portion 103 can function as an integrated hernia belt, providing abdominal support for the wearer, while the rest of the garment 100 can be of a more easy-wearing nature or well suited for physical activity.

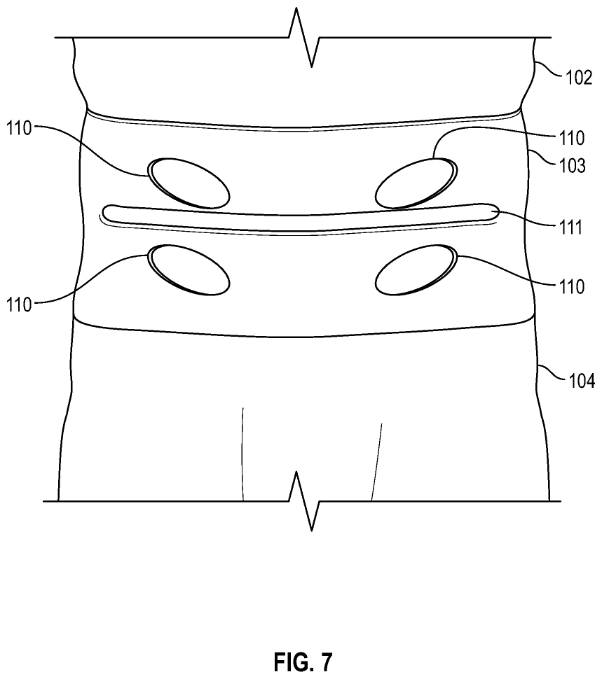

As best seen in FIGS. 4 and 7, waist portion 103 further includes one or more apertures 110 formed on the front side of the waist portion 103. The one or more apertures 110 may be formed in the part of waist portion 103 which would cover the most typical stoma placements on a wearer's abdomen. For instance, in an embodiment the waist portion 103 includes four apertures 110, two formed substantially in the middle of the lefthand side of the waist portion 103 and located vertically relative to each other, and two formed substantially in the middle of the righthand side of the waist portion 103 and located vertically relative to each other. By including multiple apertures 110 in the waist portion 103, the integrated garment 100 is readily usable by ostomates of all kinds, regardless of the exact location of their stoma. The wearer can also select the aperture that provides them with the most secure and comfortable bag placement, which can depend from wearer to wearer. In another embodiment, the garment 100 includes one aperture 110 custom placed to accommodate a particular wearer's stoma placement.

While four apertures 110 are shown in the embodiment depicted in FIGS. 4 and 7, other numbers of apertures 110 may be used. Other placements and configurations of apertures 110 in the waist portion 103 could alternatively be used to the exact placement and configuration shown in FIGS. 4 and 7. The one or more apertures 110 may be substantially oval in shape. In another embodiment, the one or more apertures 110 may be substantially circular in shape, rectangular in shape, or any other shape that readily permits access to a wearer's stoma.

To wear the garment 100, after stepping into the garment 100 and pulling the waist portion 103 over their abdomen, the wearer lines up their bag, attached to their stoma, with an aperture 110. In the case of an embodiment garment 100 featuring one aperture 110 custom placed to accommodate the particular wearer's stoma placement, the wearer uses the one aperture 110. In the case of an embodiment garment 100 with multiple apertures 110, the wearer selects the aperture that provides the wearer with the most secure and comfortable placement of their bag. For either embodiment, the wearer slides the bag through the aperture 110, making sure the aperture 110 is around the ring of the baseplate on their bag. In an embodiment, the aperture 110 is capable of stretching around the baseplate, such that a secure placement of the aperture 110 is achieved. This may be accomplished by, for instance, including a stretchable portion around the aperture 110, such as by including an elastic portion around the aperture 110.

By utilizing either a custom placed aperture 110 or including multiple apertures 110, the wearer can use an aperture 110 that provides them with the most secure and comfortable positioning of their appliance, which can be different from person to person. By achieving a secure positioning of the appliance via an aperture 110, pancake blowouts can be avoided, as a better connection of the bag to the stoma is achieved throughout the day, minimizing or eliminating stool overflow from the stoma. In another embodiment, the garment may only include one aperture 110, not custom placed for the particular wearer.

In an embodiment, the waist portion 103 further includes one or more retention elements 111 for holding in place one or more ostomy accessories. Retention element 111 may be, for example, a strap spanning substantially the width of the waist portion 103, connected by sewing or other permanent or temporary forms of connection to the waist portion 103. Retention element 111 may be connected to the waist portion 103 substantially in the middle of waist portion 103 and substantially between the two rows of apertures 110 formed in the waist portion. In another embodiment, multiple retention elements 111 may be included, for instance, by including a retention element 111 substantially higher on the waist portion 103 than the tops of the uppermost apertures 111 in addition to the retention element 111 attached substantially between the two rows of apertures 110. Retention element 111 can be used to hold the wearer's ostomy bag in place, close to the wearer's body, and/or to attach or secure various stoma accessories, such as a stoma guard, a sound proofing insert such as a muffler, or an ostomy bag cover. In this manner, the wearer does not need multiple separate belts to hold each accessory.

In an embodiment, the integrated garment 100 further includes a lower portion 104. The lower portion 104 includes at least one leg portion 104a. In an embodiment, the lower portion 104 includes two leg portions 104a. The leg portions 104a may be long (such as in pant length), mid-length (such as in shorts length or boxer length), short (such as in briefs length), or any other known leg style or configuration.

In an embodiment garment 100 that includes lower portion 104, the waist portion 103 is substantially connected to the lower portion 104, for instance, by being sewn to the lower portion 104. In an embodiment, the waist portion 103 is formed as a separate piece from the lower portion 104, which is connected to the lower portion 104, such as being sewn to the lower portion 104. The waist portion 103 may be formed from a different type of material than the type of material used to form the lower portion 104. For instance, the waist portion 103 may be formed from a type of material having compression characteristics, such as spandex, while the lower portion 104 may be formed from a type of material having comfort characteristics, such as a jersey cotton fabric, or from a type of material having wicking characteristics, such as a polyester fabric. In this manner, the waist portion 103 can function as an integrated hernia belt, providing abdominal support for the wearer, while the rest of the garment 100 can be of a more easy-wearing nature or well suited for physical activity. In another embodiment, the garment 100 does not include lower portion 104.

In an embodiment, garment 100 is formed as a one-piece integrated garment. First upper layer 101 is connected to second upper layer 102, for instance, at the shoulder seams of the two upper layers and/or the side seams of the two layers, and the waist portion 103 is connected to the second upper layer 102 and the lower portion 104. In another embodiment, the garment 100 may instead be formed as two or more separate garments, such as an upper body garment comprising the first and second upper layers 101, 102, connected to each other at the shoulder and/or side seams, and a waist portion 103 connected to the second upper layer 102; and a lower body garment that is separate from the upper body garment comprising lower portion 104. In another embodiment, the garment 100 may be instead formed as an upper body garment comprising the first and second upper layers 101, 102, connected to each other at the shoulder and/or side seams; and a lower body garment comprising waist portion 103 connected to lower portion 104.

In an embodiment garment 100, the waist portion 103 may be formed from a material different in type, texture, or other characteristic than the material from which the first and second layers 101, 102 and the lower portion 104 are formed. In an embodiment, the waist portion 103 is formed from a material having a compression characteristic while also being comfortable, such as spandex. In another embodiment, the waist portion 103 may be formed from the same type, texture, or other characteristic of material as the first and second layers 101, 102 and the lower portion 104. In another embodiment, the waist portion 103 is not formed from a separate piece of material than the second layer 103 and the lower portion 104, but rather is contiguous with the second layer 103 and the lower portion 104.

The integrated garment 100 disclosed herein can come in a variety of forms. As shown in the figures, the integrated garment 100 can be in substantially the form of a traditional union suit, having two long sleeve portions 101a, and two long leg portions legs 104a. In another embodiment, the integrated garment 100 may instead be in the form of a garment having short sleeves and short legs; no sleeves and no legs; or any other combination of sleeves (or no sleeves) and legs (or no legs).

The integrated garment 100 disclosed herein can be utilized for a variety of purposes. For instance, the integrated garment 100 may be utilized as swimwear, fitness wear, athletic wear, loungewear, daily wear, or any other conceivable purpose or activity. The integrated garment 100 may be specifically designed for men, for example, by including a fly in the lower portion 104 as is typically seen in men's briefs; or for women, for example, by including an opening with a closure in the crotch region of the lower portion 104 so that the suit does not have to be removed to urinate for a colostomy or ileostomy and/or by including a compression portion in the second upper layer 102 to support breasts. Alternatively, the integrated garment may be designed unisex, i.e., not specifically designed for a man or a woman. Whether the garment is designed specifically for a man or a woman or is unisex, the garment may additionally include an opening with closure in the rear seat area of the lower portion 104 so that the suit does not have to be removed for a bowel movement for persons who have had a urostomy rather than a colostomy or ileostomy.

The integrated garment 100 may include additional or different features depending on the nature of the garment's intended use. For example, in an embodiment garment 100 intended to be utilized as swimwear, instead of including plackets 112, 114 located on the front side of the first and second upper layers 101, 102, the garment 100 may instead include a zipper or other garment closure type located in the center rear side of the first and second upper layers 101, 102, formed at the necklines of the first and second upper layers 101, 102. This zipper would, like the plackets previously discussed, enable the wearer to open the garment to step into the garment and pull the garment up, such that the waist portion 103 is positioned substantially around the wearer's midsection. The first and second upper layers 101, 102 may be connected to each other, for instance, at the top shoulder seams and/or the side seams. The first upper layer 101 may have a bit of an “untucked” look on at least one side, permitting the bag to fall naturally as it fills. The side openings 107 may include waterproof or water-resistant zipper closures or other waterproof or water-resistant closure types to permit protection of the ostomy bag inside the internal pocket 105 when the garment is worn in the water. The swim garment 100 may be as otherwise described herein; for instance, the first upper layer 101 includes at least one shoulder portion 101a and may include at least one sleeve portion 101b; the second upper layer 102 includes at least one shoulder portion 102a; the lower portion 104 may include at least one leg portion 104a, which could be in the style of board shorts; and the waist portion 103 includes one or more apertures 110.

The integrated garment 100 disclosed herein eliminates the need for an ostomate to wear multiple specialized belts, shields, and garments in order to accommodate the ostomate's appliances. By offering an integrated garment 100, ostomates are provided with a level of comfort, security, and confidence not previously available. Further, by integrating the garment 100 as disclosed herein, numerous problems seen in existing belts and garments are reduced or eliminated. The integrated hernia belt does not roll or otherwise shift; the ostomy bag need not be attached to the wearer's skin or otherwise be in contact with the wearer's skin; the weight of the ostomy bag does not pull down on the wearer's stoma; the garment need not be removed to go to the bathroom or otherwise access or replace the ostomy bag; and multiple accessories can be held in place by one comprehensive garment. In this manner, ostomates can continue to live a normal, active life, allowing them to retain their sense of self in their next chapter of life.

This written description makes reference to particular features. It is to be understood that the disclosure in this specification includes all possible combinations of those particular features. For example, where a particular feature is disclosed in the context of a particular example configuration, that feature can also be used, to the extent possible, in the context of other example configurations.

Also, when reference is made in this application to a method having two or more defined steps or operations, the defined steps or operations can be carried out in any order or simultaneously, unless the context excludes those possibilities.

Furthermore, the terms “comprises” and “includes” and their grammatical equivalents are used in this application to mean that other components, features, steps, processes, operations, etc. are optionally present. For example, an article “comprising” or “which comprises” components A, B, and C can contain only components A, B, and C, or it can contain components A, B, and C along with one or more other components. Likewise, an article “including” or “which includes” components A, B, and C can contain only components A, B, and C, or it can contain components A, B, and C along with one or more other components.

Although specific example configurations have been described for purposes of illustration, it will be understood that various modifications may be made without departing from the spirit and scope of the disclosure.

What is claimed is:

1. An integrated ostomy garment comprising:
a first upper layer, the first upper layer including at least one shoulder portion, the first upper layer further including an internal pocket formed on an internal front side of the first upper layer, the internal pocket having a top opening;
a second upper layer, the second upper layer including at least one shoulder portion;
a lower portion; and
a waist portion, the waist portion substantially connected to the second upper layer and the lower portion, the waist portion including at least one aperture formed on a front side of the waist portion.

2. The integrated ostomy garment of claim 1, wherein the first upper layer is substantially connected to the second upper layer.

3. The integrated ostomy garment of claim 1, the first upper layer further including a side opening by which the internal pocket may be accessed externally.

4. The integrated ostomy garment of claim 1, the first upper layer further including a fastening apparatus consisting of a first closure element located substantially in the middle of an external front side of the first upper layer, and a second closure mechanism located substantially in the middle of a lower hemline of the first upper layer.

5. The integrated ostomy garment of claim 4, wherein the fastening apparatus consists of a button and a corresponding buttonhole.

6. The integrated ostomy garment of claim 1, the first upper layer further including at least one sleeve portion.

7. The integrated ostomy garment of claim 1, wherein the internal pocket further includes a retention element internal to the internal pocket.

8. The integrated ostomy garment of claim 1, wherein the internal pocket further includes a removable liner.

9. The integrated ostomy garment of claim 1, wherein the first upper layer further includes a placket formed at a neckline of the first upper layer and substantially in the middle of the front side of the first upper layer.

10. The integrated ostomy garment of claim 1, wherein the waist portion further includes a retention element connected to an external front side of the waist portion.

11. The integrated ostomy garment of claim 1, wherein the waist portion is formed of a material having compression characteristics.

12. The integrated ostomy garment of claim 1, wherein the lower portion further includes at least one leg portion.

13. The integrated ostomy garment of claim 1, wherein the second upper layer further includes a placket formed at a neckline of the second upper layer and substantially in the middle of a front side of the second upper layer.

14. The integrated ostomy garment of claim 1, wherein the waist portion is formed of a material type that is different than a material type form which the first upper layer, the second upper layer, and the lower portion are formed.

15. An integrated ostomy garment comprising:
a first upper layer, the first upper layer including two shoulder portions, the first upper layer further including an internal pocket formed on an internal front side of the first upper layer, the internal pocket having a top opening, the first upper layer further including a first side opening formed substantially on the lefthand side of the first upper layer by which the internal pocket may be accessed externally to the garment, and a second side opening formed substantially on the righthand side of the first upper layer by which the internal pocket may be accessed externally to the garment;
a second upper layer, the second upper layer including two shoulder portions, the second layer being substantially connected to the first upper layer;
a lower portion; and
a waist portion, the waist portion substantially connected to the second upper layer and the lower portion, the waist portion including an aperture formed on a front side of the waist portion, the waist portion further including a retention element connected to an external front side of the waist portion;
wherein the waist portion is formed of a material having compression characteristics, the material from which the waist portion is formed being different in type than a material type from which the first upper layer, the second upper layer, and the lower portion are formed.

16. The integrated ostomy garment of claim 15, the first upper layer further including a fastening apparatus consisting of a first closure element located substantially in the middle of an external front side of the first upper layer, and a second closure element located substantially in the middle of a lower hemline of the first upper layer.

17. The integrated ostomy garment of claim 16, wherein the fastening apparatus consists of a button and a corresponding buttonhole.

18. The integrated ostomy garment of claim 15, the first upper layer further including two sleeve portions, and the lower portion further including two leg portions.

19. The integrated ostomy garment of claim 15, wherein the internal pocket has two top openings, one formed substantially on the lefthand side of the internal pocket, and one formed substantially on the righthand side of the internal pocket.

20. The integrated ostomy garment of claim 15, wherein the first upper layer further includes a placket formed at a neckline of the first upper layer and substantially in the middle of the front side of the first upper layer, and the second upper layer further includes a placket formed at a neckline of the second upper layer and substantially in the middle of a front side of the second upper layer.
